(19) United States
(12) Patent Application Publication
(10) Pub. No.: US 2008/0293079 A1
(43) Pub. Date: Nov. 27, 2008

(54) Chip for Diagnosing the Presence of Candida

(75) Inventors: Nicole Hauser, Herrenberg (DE); Steffen Rupp, Stuttgart (DE); Achim Weber, Altbach (DE); Guenter Tovar, Stuttgart (DE); Ekkehard Hiller, Stuttgart (DE); Kirsten Borchers, Stuttgart (DE)

Correspondence Address: DARBY & DARBY P.C., P.O. BOX 770, Church Street Station, New York, NY, 10008-0770, US

(73) Assignees: Frauhofer-Gesellschaft Zur Foerderung Der Angewandtten Forschung e.V., Muenchen (DE); Universitaet Stuttgart, Stuttgart (DE)

(21) Appl. No.: 12/066,136

(22) PCT Filed: Sep. 27, 2006
(86) PCT No.: PCT/EP2006/009363

§ 371 Date: May 18, 2008

(30) Foreign Application Priority Data
Sep. 28, 2005 (DE) 102005047384.9

(51) Int. Cl. G01N 33/569; C12M 1/34; B05D 1/00

(52) U.S. Cl. 435/7.31; 435/287.2; 427/2.13

(57) ABSTRACT

The present invention concerns means and methods of detection of Candida and Candida-related fungal cells in clinical material by means of protein biochips.

This application is a U.S. national phase application under 35 U.S.C. §371 of International Patent Application No. PCT/EP2006/009363 filed Sep. 27, 2006, which claims the benefit of priority to German Patent Application No. DE 10 2005 047 384.9 filed Sep. 28, 2005, the disclosures of all of which are hereby incorporated by reference in their entireties. The International Application was published in German on Apr. 5, 2007 as WO 2007/036352.

### FIELD OF THE INVENTION

The present invention concerns means and methods of detection of Candida and Candida-related fungal cells in clinical material.

### BACKGROUND

Candida albicans is a fungus of the Candida group, which belong to the yeast fungi. This fungus is often to be found on the mucous membranes of the nose and throat and in the genital region, as well as in the digestive canal of warm-blooded animals (and therefore also man). It can be detected in around 75% of all healthy men and women (according to the German Nutrition Society). It can also occur between fingers and toes and on fingernails and toenails. Candida is one of the facultative pathogens (causing an illness only under certain circumstances) and is considered to be a saprophyte, living in a state of equilibrium with other microorganisms. Generally, colonization by this fungus does not cause any symptoms. However, if the immunity is reduced or deficient, as in the case of other underlying diseases and/or when taking medication, these fungi become pathogenic germs. A Candida infection will occur, such as candidosis, candidiasis, candidamycosis, monoliasis or thrush.

Usually, a Candida infection occurs during underlying diseases such as severe diabetes, leukemia, AIDS, under the action of certain medications such as contraceptives, medications which lower the resistance deliberately or as a side effect, antibiotics when taken frequently and in high doses, corticoids and cytostatics in high doses, and/or other favorable circumstances. The risk groups include tumor patients with neutropenia, patients after bone marrow transplantation or other organ transplantation, immunosuppressed patients, patients with large wound areas or burns, polytraumatized patients and the newborn. Furthermore, there are predisposing factors for a systemic Candida infection in intensive care patients.

The actual pathophysiological mechanism which leads to the formation of a deep candidosis and subsequently to life-threatening Candida sepsis is not yet clearly explained. The tissue-damaging action comes primarily from toxic, still little understood fungal products.

The incidence of candidemia and dessiminated candidiasis in intensive care patients has increased considerably in recent years. Given the associated high morbidity and mortality, it would be desirable to overcome the difficulties of the diagnosis with definite and early detection of the Candida invasion.

The diagnosis of a candidiasis in the routine clinical laboratory is mostly done by microscope. Mucous membrane swabs, stool samples, urine, a positive blood culture or other investigatory material from sterile organ compartments (spinal fluid, tissue biopsy) can be suitable. In this case, certain detection of a candidiasis only seldom occurs. In any case, false positive results are frequent, while false negative findings can even occur during thrush sepsis. Furthermore, the culturing of patient samples is very time intensive, and therefore often the diagnosis is made too late.

Fungi are living antigen mosaics and can stimulate the different parts of the immune system. Antigens of the fungal capsule in the form of proteins, polysaccharides, lipids and chitin-like substances induce an antibody formation by B-cells. As a result, corresponding precipitating and complement-binding antibodies can be detected in the serum of fungus-infected patients. Given clinical suspicion of a systemic Candida infection, serological investigations of the course of the disease will often show a simultaneous rise in the titer of antibodies directed against Candida.

Known antibody assays are based on antibodies against cell wall proteins, which are usually immobilized on substrate spheres (so-called “beads”). Clinical samples such as blood are brought into contact with the antibody beads in an arrangement similar to a blood group determination. If Candida-specific cell wall components are present in the sample, there will be a clumping of the beads, which becomes visible in a cavity plate or a microtitration plate. This test is known as the so-called hemagglutinin test (HAT). But these tests are greatly debated in medicine, owing to their poor sensitivity and informativeness.

An effective, life-saving treatment could occur more quickly and specifically thanks to a fast, accurate, and more informative detection of this infection. The success of a fungal therapy depends considerably on how timely the therapy is initiated. On the other hand, the antimycotics used have not insignificant side effects. Although special, newly developed and highly effective antimycotics have fewer side effects, they are also much more costly in their application. Besides a fast and sensitive detection of Candida, a Candida test should thus also have a high selectivity, in order to minimize the number of false positive results and, thus, the number of needless therapies.

Moreover, a Candida test should be fast and safe to use in routine clinical diagnostics. This means that, with reduced costs for the individual test and low expense for specialized personnel, it must make possible the highest possible specimen processing rate. This can generally be achieved by the use of automated reading instruments, which in particular are in direct connection with the patient's databases. Ideally, a high number of individual tests should be accomplished in a single run-through. Moreover, an improved test must offer the possibility of being carried out in a single batch with other tests used, for example, to detect other pathogens.

### DETAILED DESCRIPTION OF THE INVENTION

The present invention is based on the technical problem of providing means and methods for the detection of Candida and Candida-related fungal cells in clinical material, where the drawbacks known in the prior art are eliminated. In particular, an enhanced sensitivity and selectivity will be achieved, and which are suitable for use in automated screening and analysis systems.

The present invention solves its underlying technical problem by the providing of a functional element for the detection of Candida, that is, a Candida diagnosis chip, comprising a substrate with a surface and at least one microstructure arranged on the substrate surface with molecule-specific recognition sites, chosen from among: specific antibodies against protein TSA 1, preferably so-called anti-TSA 1 IgG, and protein TSA 1, which are immobilized thereon.

By TSA is meant here the “Thiol-specific-antioxidant-(iike) protein” of Candida, a member of the peroxiredoxin enzyme family (EC 1.11.1.15). This is a physiologically important antioxidant with disulfide bond, which can fight off sulfur-containing radicals by means of enzymatic activity. TSA 1 is primarily localized in the cytosol. TSA 1 has the amino acid sequence SEQ ID NO: 1.

Preferably, TSA 1 is used in the form of recombinant TSA 1. Of course, a fragment or a derivative of TSA 1 can be used according to the invention. The fragment or the derivative can be obtained by exchange and/or omission of 1, 2, 3, 4, 5, 6, 7, 8, 9, 10, 1 to 10, 1 to 20, 1 to 30, 1 to 40, and/or 1 to 50 amino acids from the protein per SEQ ID NO: 1. The fragment or the derivative of TSA 1 also has Candida-specific antigenicity and specifically binds to Candida-specific anti-TSA 1 antibodies (anti-TSA 1 IgG). In another variant preferred by the invention, the TSA 1 protein is part of a Candida cell lysate or a protein cocktail, which is obtained from Candida cells, such as cytosol proteins or cell wall proteins. The functional element then also has the molecule-specific recognition sites of the invention, if yet additional Candida proteins are immobilized besides the TSA 1 protein.

Preferably, the microstructure is formed from several three dimensionally superimposed layers of nanoparticles and the nanoparticles have the molecule-specific recognition sites.

Further preferred are microstructures that have identical molecule-specific recognition sites for Candida antigens or antibodies. Further preferred are microstructures which also have nonidentical molecule-specific recognition sites for Candida antigens or antibodies. These structures make it possible to integrate several different Candida proteins in a single test.

In a preferred embodiment, the microstructures are formed with inclusion of at least one biomolecule stabilizing agent. The layers of nanoparticles, preferably in multidimensional arrangement, drastically increase the reaction surfaces of the functional element available for the desired detection reactions, while at the same time in a preferred embodiment when using TSA 1 protein or anti-TSA 1 antibodies the natural structure and function of the proteins is preserved thanks to the inclusion of the protein-stabilizing agent.

Preferably, the several preferably three-dimensionally arranged layers of nanoparticles are arranged in a thickness of 10 nm to 10 μm, preferably 50 nm to 2.5 μm, especially preferably 100 nm to 1.5 μm on the substrate surface. The makeup of the functional element according to the invention enables a high sensitivity of detection, even for the smallest quantities of analytes being detected.

Preferably, the functional elements used according to the invention for the detection of Candida—in lateral structuring—are outfitted with other functional layers with different molecule-specific recognition sites, each of them being specifically addressable. Thus, specific locally detached analytes can be bound. The parallel detection of analytes other than Candida-specific molecules on a single functional element, in a single detection method, is made possible in this way. Additional molecule-specific recognition sites—depending on the area of application—are preferably proteins and/or antibodies that are used specifically for microbial pathogens such as fungal cells; preferably the fungal cells are clinically relevant pathogens such as Aspergillus, Cryptococcus (Histoplasma, Blastomyces), Coccidioides immitis, Epidermophyton, Geotrichium, Paracoccidioides (Blastomyces). Other molecule-specific recognition sites are preferably other selected isolated Candida antigens and/or antibodies directed against other Candida antigens.

Thus, the present invention provides a functional element, on whose surface one or more microstructures are arranged, while each microstructure preferably consists of many nanoparticles, especially preferably in several layers with identical or nonidentical molecule-specific recognition sites, wherein at least one molecule-specific recognition site is chosen from among: specific antibodies to the protein TSA 1, preferably so-called anti-TSA 1 IgG, and the protein TSA 1.

Contrary to the systems known in the prior art, such as traditional gene or protein arrays, the present invention thus calls for binding biological molecules not directly on a planar surface, but instead to immobilize them on several, preferably three-dimensional, nanoparticle surfaces, which are used to form a laterally structured microstructure before or after the immobilization.

On the functional elements of the invention, the molecule-specific recognition sites are covalently and/or noncovalently bound to the nanoparticles. The specific antibodies to the protein TSA, or the protein TSA, can be immobilized nondirectionally as well as directionally on the nanoparticles, while almost any desired orientation of the biomolecules is possible. Thanks to the immobilization of the biomolecules on the nanoparticles, a stabilization of the biomolecules is also achieved.

In the context of the present invention, by a “nanoparticle” is meant a particulate binding matrix, which has molecule-specific recognition sites comprising first functional chemical groups. The nanoparticles used according to the invention comprise a core with a surface, on which the first functional groups are arranged, being able to bind covalently or noncovalently to complementary second functional groups of a biomolecule. Thanks to the interaction between the first and second functional groups, the biomolecule is immobilized and/or can be immobilized on the nanoparticle and thus on the microstructure of the functional element. The nanoparticles used according to the invention to form the microstructures have a size less than 500 nm, preferably less than 150 nm.

The nanoparticles preferably used according to the invention have a core and shell structure. In preferred embodiments, the core of the nanoparticles consists of an inorganic material, such as a metal, for example, Au, Ag or Ni, silicon, SiO2, SiO, a silicate, Al2O3, SiO2.Al2O3, Fe2O3, Ag2O, TiO2, ZrO2, Zr2O3, Ta2O5, zeolite, glass, indium tin oxide, hydroxyl apatite, a Qdot or a mixture thereof, or it contains these. In other preferred embodiments, the core consists of an organic material or contains this. Preferably, the organic polymer is polypropylene, polystyrene, polyacrylate, a polyester of lactic acid or a mixture thereof. The preparation of the cores of the nanoparticles used according to the invention can take place by using customary, known techniques of this special field, such as sol-gel synthesis methods, emulsion polymerization, suspension polymerization, etc.

In a preferred embodiment, additional functions are anchored in the core, making possible a simple detection of the nanoparticle cores and, thus, the microstructures by use of suitable detection methods. These functions can be, for example, fluorescence markings, UV/V is markings, superparamagnetic functions, ferromagnetic functions and/or radioactive markings. Suitable methods for the detection of nanoparticles constitute, for example, fluorescence and/or UV-Vis spectroscopy, fluorescence or light microscopy, impedance spectroscopy, electrical and radiometric methods. Also, a combination of the methods can be used for the detection of the nanoparticles. In another embodiment, the core surface can be modified by emplacing additional functions such as fluorescence markings, UV/Vis markings, superparamagnetic functions, ferromagnetic functions, and/or radioactive markings. Preferably, the surface of the nanoparticle cores has ion exchange functions, separately or in addition. Nanoparticles with ion exchange functions are especially suitable for optimization of MALDI analysis, since they can bind to disruptive ions.

Moreover, it is provided that the core surface has chemical compounds which serve for the steric stabilization and/or to prevent a conformational change of the immobilized molecules and/or to prevent the build-up of other biologically active compounds on the core surface. Preferably, these chemical compounds are polyethylene glycols, oligoethylene glycols, dextran or a mixture thereof.

Nanoparticles used preferably according to the invention have a diameter of 5 nm to 500 nm. By using such nanoparticles, therefore, one can prepare functional elements that have very small microstructures of any desired shape in the nanometer to micrometer region. The use of the nanoparticles to create the microstructures therefore allows a heretofore unachieved miniaturization of the functional elements, which is accompanied by substantial improvements of significant parameters of the functional elements.

By a “microstructure” is meant structures in the region of a few micrometers or nanometers. In particular, in the context of the present invention, “microstructure” means a structure which consists of at least two individual components in the form of several three-dimensionally arranged layers of nanoparticles with molecule-specific recognition sites and is arranged on the surface of a substrate, while a certain surface segment of the surface of the substrate is covered, having a definite shape and a definite surface content and being smaller than the substrate surface. According to the invention, it is provided in particular that at least one of the surface/length parameters that dictates the surface segment covered by the microstructure lies in the micrometer region. For example, if the microstructure has the shape of a circle, the diameter of the circle lies in the micrometer region. If the microstructure is designed as a rectangle, for example, the width of this rectangle lies in the micrometer region. In particular, it is provided according to the invention that the at least one surface/length parameter that dictates the surface segment covered by the microstructure is smaller than 999 μm. Since the microstructure according to the invention consists of at least two nanoparticles, the lower limit of this surface/length parameter lies at 10 nm.

In one preferred embodiment, three-dimensionally arranged layers of nanoparticles have an overall thickness of 10 nm to 10 μm. According to the invention, a thickness of 50 nm to 2.5 μm, but especially a thickness of 100 nm to 1.5 μm, is preferred.

The nanoparticles used preferably for the formation of the microstructures possess a relatively very large surface/volume ratio and accordingly can bind a large amount of a biological molecule per mass. As compared to systems in which biological molecules are bound directly to a planar substrate, a functional element can thus bind a sizeably larger amount of the biological molecules per unit of surface. The amount of molecules bound per unit of surface, that is, the packing density, is so large, according to the invention, because several layers of particles are layered one on top of the other to create the microstructure on the substrate surface. A further increasing of the amount of biological molecules bound per unit of surface is preferably achieved in that the nanoparticles are first coated with hydrogels and then with the biological molecules.

In the context of the present invention, by “functional element” is meant an element that performs at least one definite function either alone or as part of a more complex device, that is, in conjunction with other similar or differently constituted functional elements. A functional element comprises several components, which can consist of the same or different materials. The individual components of a functional element can perform different functions within a functional element and can contribute to the overall function of the element in differing degree or in different manner and kind. In the present invention, a functional element comprises a substrate with a substrate surface, on which defined layers of nanoparticles are arranged preferably three-dimensionally as microstructure(s), while the nanoparticles are provided with molecule-specific recognition sites chosen from among: specific antibodies against the protein TSA 1, preferably so-called anti-TSA 1 IgG, and the protein TSA 1, for the binding of Candida-specific molecules.

The functional elements of the invention can be prepared in simple manner by using known methods. For the preparation and for further embodiments of the function elements, refer to later published German patent application DE 10 2004 062 573, whose disclosure content is incorporated here in its full extent.

For example, by using suitable suspension agents, stable suspensions can be created very easily from nanoparticles. Nanoparticle suspensions behave like solutions and are in this way compatible with microstructuring processes. Therefore, nanoparticle suspensions can be deposited in structured manner directly onto substrates previously treated with a bonding agent for firm adhesion of the nanoparticles, such as by using traditional methods like needle-ring printers, lithographic processes, ink jet processes and/or microcontact methods. Thanks to a suitable choice of the bonding agent, the microstructure formed can be shaped so that at a later time it can be detached in part or entirely from the substrate surface of the functional element, for example, by altering the pH value or the temperature, and be transferred if desired to the substrate surface of another functional element.

Preferably according to the invention at least one biomolecule-stabilizing agent, especially at least one protein-stabilizing agent, is enclosed in the microstructure. Thanks to such agents, the stabilization of the biomolecules is further strengthened. The addition of at least one biomolecule-stabilizing additive, especially at least one protein-stabilizing additive, preserves the functionality of nanoparticle-bound biological molecules, especially peptides or proteins, within the particle layers, when these are dried onto a substrate, and thus guarantees the shelf life of nanoparticulate functional layers. The shelf life is thus up to one year, preferably up to 8 months, in particular 3 months. The inclusion of at least one biomolecule-stabilizing agent according to the invention, in particular, at least one protein-stabilizing agent in the microstructure thus protects the function, primarily the biological function, and the efficacy of the invented functional elements. By “biomolecule-stabilizing agents” and especially “protein-stabilizing agents” is meant, according to the invention, agents which stabilize the three dimensional structure of proteins, i.e., the secondary, tertiary and quaternary structure, under drying stress, and thereby preserve the functionality of the proteins in the dry state, that is, after the solvent is evaporated off. In one preferred embodiment, the protein-stabilizing agent is a saccharide, especially saccharose (sucrose), lactose, glucose, trehalose or maltose, a polyalcohol, especially inositol, ethylene glycol, glycerol, sorbitol, xylitol, mannitol or 2-methyl-2,4-pentane diol, an amino acid, especially sodium glutamate, proline, alpha-alanine, beta-alanine, glycine, lysine-HCl or 4-hydroxyproline, a polymer, especially polyethylene glycol, dextran, polyvinyl pyrrolidone, an inorganic salt, especially sodium sulfate, ammonium sulfate, potassium phosphate, magnesium sulfate or sodium fluoride, an organic salt, especially sodium acetate, sodium polyethylene, sodium caprylate, propionate, lactate or succinate, or trimethylamine N-oxide, sarcosin, betaine, gamma-aminobutyric acid, octopin, analopin, strombin, dimethyl sulfoxide or ethanol, or a mixture of the mentioned substances.

According to the invention, the substrate of the functional element, especially the substrate surface, consists of a metal, a metal oxide, a polymer, glass, a semiconductor material or ceramic. In preferred embodiment, the substrate of the functional element consists of materials such as transparent glass, silicon dioxide, metals, metal oxides, polymers and copolymers of dextrans or amides, such as acrylamide derivatives, cellulose, nylon, or polymer materials, such as polyethylene terephthalate, cellulose acetate, polystyrene or polymethylmethacrylate or a polycarbonate of bisphenol A. In the context of the invention, this means that either the substrate consists entirely of one of the above mentioned materials or essentially contains it. The substrate or its surface will consist of at least around 60%, preferably around 70%, around 80%, or around 100% of one of the above mentioned materials or a combination of such materials.

In preferred embodiment of the invention, at least one layer of a bonding agent is arranged between the substrate surface and the microstructure. The bonding agent serves for a firm bonding of the nanoparticles to the substrate surface of the functional element. The choice of the bonding agent will depend on the surface of the substrate material and the nanoparticles being bound. The bonding agent is preferably charged or uncharged polymers. The bonding agents are preferably weak or strong polyelectrolytes, that is, their charge density is pH-dependent or pH-independent. In one preferred embodiment, the bonding agent consists of poly(diallyl-dimethyl-ammonium chloride), a sodium salt of poly(styrene sulfonic acid), a sodium salt of poly(vinylsulfonic acid), poly(allylamino-hydrochloride), linear or branched poly(ethylene imine), poly(acrylic acid), poly(methacrylic acid) or a mixture of these. The polymer is preferably a hydrogel.

Other preferred bonding agents are chosen from functional silanes, especially for the activation of glass surfaces, silicon surfaces or the like, and functional thiols, especially for the activation of gold surfaces. These molecules essentially consist of an “anchor”, such as silanol, chlorsilane or the like, a “spacer”, such as polyethylene glycol, oligoethylene glycol, hydrocarbon chains, carbohydrate chains, or the like, and at least one functional group, preferably an amino group, carboxy group, hydroxy group, epoxy group, tosyl chloride, N-hydroxy-succinimide ester, maleimide and/or biotin.

Other preferred bonding agents are also polymers that contain active esters, such as phenyldimethyl-sulfonium methyl sulfate groups, photoactive cross-linkers, proteins like streptavidine, BSA and the like, as well as nucleic acids.

Combinations of at least two of the mentioned bonding agents are also preferred.

In the context of the present invention, “addressable” means that the microstructure after the deposition of the nanoparticles onto the substrate surface can once again be found and/or detected. For example, if the microstructure is deposited by using a mask or an upper die onto the substrate surface, the address of the microstructure results, on the one hand, from the x and y coordinates of the region of the substrate surface dictated by the mask or the die, on which the microstructure has been deposited. On the other hand, the address of the microstructure results from the molecule-specific recognition sites on the surface of the nanoparticles, which enable a retrieval or a detection of the microstructure.

The present invention, moreover, concerns the use of the invented functional element for the detection of Candida and Candida-related fungal cells, i.e., especially for the diagnosis of candidoses in human or animal bodies.

By “clinical material” or “sample of a clinical material” is meant a sample such as whole blood, blood serum, lymph, tissue fluid, bronchial lavage, gastrointestinal rinse liquid, stool, cervical mucus, or a mucous membrane swab. It also means a biopsy or tissue sample taken from a living or dead organism, organ or tissue. But a sample can also be a culture medium, for example, a fermentation medium, in which organisms such as microorganisms, or human, animal or plant cells have been cultivated. Such a sample can already have undergone purification steps, such as protein isolation, or it can also be unpurified.

The invented use of the invented functional element makes use of the specific antigen/antibody binding between the molecule-specific recognition sites, chosen from among specific antibodies to the protein TSA 1 and the protein TSA 1, with corresponding Candida-specific molecules occurring in the sample of clinical material being investigated.

The antigen/antibody complex resulting from the functional element making contact with the provided clinical material can be detected in familiar fashion. Known methods of immunohistology, appropriately adopted, can be applied to the functional elements. Preferably according to the invention labeled antigen proteins or labeled primary or labeled secondary antibodies are used for the detection of antigen/antibody complex on the functional element, which label the Candida-specific molecules of the sample that are specifically bound in the antigen/antibody complex by a further specific antigen/antibody binding. The labeling agent preferably used is fluorescence labeling or metal labeling. To detect this labeling, MALDI mass spectrometry, fluorescent or UV-VIS spectroscopy, fluorescent or light microscopy, waveguide spectroscopy, electrical methods such as impedance spectroscopy, or a combination of these methods are preferably used.

If a fluorescent detection method is used, a fluorescently labeled analyte and/or fluorescently labeled detection molecule that is biologically active and bound to the nanoparticle is excited by light and read using light. Preferably according to the invention when using a fluorescence method, the analyte and/or the molecule-specific detection molecule and/or another secondary detection molecule, such as a secondary antibody, streptavidine, etc., is fluorescently labeled.

Especially preferably, the detection of the labeled antigen/antibody complex takes place automatically, for example, in scanners.

The present invention therefore also concerns a method for identification and/or for detection of Candida and Candida-related fungal cells, especially in clinical material, i.e., especially a method for the diagnosis of candidoses in human or animal bodies. In one step a) of the method, a sample, especially one of clinical material, is made ready. In another step b) of the method, a functional element according to the invention, i.e., a Candida diagnosis chip, is prepared, and this is brought into contact in another step c) of the method with the sample under conditions which make possible a specific antigen/antibody binding, wherein Candida-specific molecules from the sample are bound to the molecule-specific recognition sites of the functional element, chosen from among specific antibodies to the protein TSA 1, and the protein TSA 1, in an antigen/antibody complex. In another step f) of the method, the antigen/antibody complex formed on the Candida diagnosis chip is detected in familiar fashion, preferably by means of fluorescently labeled antigens or antibodies. In step e), therefore, the Candida-specific molecules bound on the Candida diagnosis chip are preferably bound with fluorescently labeled molecules, such as labeled antibodies, labeled secondary antibodies, labeled recombinant proteins, etc. In another preferred form of the method, a MALDI mass spectrometry method is adopted as the detection method.

Preferably, after step c) and before the detection in step f), nonbound Candida-specific molecules and also nonspecific molecules are removed from the functional element by washing with a biocompatible washing liquid in an additional step d). The biocompatible washing liquid is preferably water and/or buffer, such as phosphate-buffered saline (PBS) and/or buffer with addition of a detergent, such as TritonX-100. In a preferred embodiment of the invention, the substrate is washed at room temperature sequentially in water and buffer, with a detergent if desired, or buffer, with a detergent if desired, and water, for example, 30 min for each.

Another use of the functional element according to the invention is the isolation of a protein from a sample that enters into interaction with the immobilized molecule-specific recognition sites, chosen from among specific antibodies to the TSA 1 protein and the TSA 1 protein.

Finally, the present invention also concerns the use of the functional element for the development and production of pharmaceutical products for the diagnosis and treatment of candidoses and related fungal infections of the human or animal body.

Other beneficial embodiments of the invention will result from the subclaims.

The sequence protocol contains:

SEQ ID NO: 1 amino acid sequence of TSA 1 (Candida albicans).
SEQ ID NO: 2 amino acid sequence of the binding sequence of a polyclonal antibody used.
SEQ ID NO: 3 amino acid sequence of the binding sequence of a polyclonal antibody used.
SEQ ID NO: 4-amino acid sequence of TSA 1-MBP fusion protein.
SEQ ID NO: 5 amino acid sequence of MBP.
SEQ ID NO: 6-amino acid sequence of the linker for TSA 1 at the C-terminal end of MBP.

The invention shall now be explained more closely by means of the following figures and examples.

### BRIEF DESCRIPTION OF THE DRAWINGS

FIG. 1 shows the outcome of the detection of rabbit anti-TSA 1 antibodies. The antibody (35 ng/ml) is detected by means of nanoparticulate affinity layers. The sensor layers consist of functional nanoparticles which have Candida cell lysate bound to their surface. The detection of the binding occurs through a fluorescently labeled anti-rabbit antibody.

FIG. 2 shows the outcome of the detection of fluorescently labeled Candida antigen. The recombinant antigen is detected by means of nanoparticulate affinity layers in a concentration of 40 μmol/l. The sensor layers consist of functional nanoparticles which have anti-TSA 1 antibodies bound to their surface.

FIG. 3 shows the outcome of the detection of Candida antigen by means of the sandwich technique. The recombinant antigen is detected by means of nanoparticulate affinity layers in a concentration of 100 μmol/l. The sensor layers consist of functional nanoparticles which have anti-TSA 1 antibodies bound to their surface. The detection occurs via a fluorescently labeled anti-TSA 1 antibody.

### EXAMPLES

### Example 1

### Detection of Anti-Candida albicans Antibodies in Clinical Material

In this example, an antibody is detected that is directed against the antigen TSA 1 of Candida albicans. The detection of anti-Candida antibodies in a sample is done by immobilizing Candida cell lysate on functional silica nanoparticles and depositing these bioactive nanoparticles as an affinity coating on a substrate. The anti-Candida antibodies present in the sample bind to Candida antigen TSA, which is immobilized in three dimensionally nanostructured affinity layers. The detection of the binding was done by means of fluorescently labeled secondary antibody.

### 1.1 Preparation of Nanoparticle-Based Candida Diagnosis Chips

### Substrate:

In order to prepare nanoparticle-based Candida diagnosis chips that are suitable for fluorescence reading, one uses glass substrates, for example. The adhesion of the nanoparticles to surfaces is for the most part mediated by electrostatic interaction in this case. One usually requires positively charged surfaces for the adsorption of protein-coated nanoparticles on the substrate. Commercially available glass specimen slides, which have positive groups on the surfaces, are imprinted with protein-coated nanoparticles with no other pretreatment.

Traditional glass surfaces are cleaned in a 2 vol. % aqueous HELLMANEX solution for 90 minutes at 40 degrees C. After washing in MilliQ-H2O (deionized water, 18 MOhms), the glass specimen slides are hydroxylated in a 3:1 (v/v) NH3/H2O₂ solution for 40 min at 70 degrees C. (NH3 puriss. p.a., around 25% in water, and H₂O2 for analysis, 30%, ISO Reag., stabilized).

After thorough washing in MilliQ water, the substrates are incubated for 20 min at room temperature in an aqueous polycation solution (0.02 mol/l poly(allylamine) (in terms of the monomer), pH 8.5), washed for 5 min in MilliQ water, and then dried by centrifuging.

### Synthesis of Core/Shell Particles:

To 200 ml of ethanol, one adds 12 mmol of tetraethoxysilane and 90 mmol of NH3. One then stirs for 24 h at room temperature. After this, the particles are cleaned by multiple centrifuging. The result is 650 mg of core and shall particles with a mean particle size of 125 nm.

### Amino Functionalization of Core/Shell Particles:

A 1 wt. % aqueous suspension of the core and shell particles is reacted with 10 vol. % ammonia. Then, 20 wt. % of aminopropyltriethoxysilane, in terms of the particles, is added and one stirs for 1 h at room temperature. The particles are cleaned by multiple centrifuging and bear functional amino groups on their surface (zeta potential in 0.1 mol/l acetate buffer: +35 mV).

### Carboxy Functionalization of Core/Shell Particles

Ten milliliters of a 2 wt. % suspension of amino functionalized nanoparticles are taken up in tetrahydrofuran. To this one adds 260 mg of succinic acid anhydride. After a 5 min treatment with ultrasound, one stirs for 1 h at room temperature. The particles are cleaned by multiple centrifuging and bear functional carboxy groups on their surface (zeta potential in 0.1 mol/l acetate buffer: −35 mV). The mean particle size is 170 nm.

### 1.2 Binding of the Molecule-Specific Recognition Sites to the Core/Shell Particles—Binding of TSA 1 Protein Containing Candida Cell Lysate

One milligram of carboxy-functionalized core and shell particles is combined with 30 μl of a Candida cell lysate, which contains the antigen TSA 1, and 10 μl of an EDC solution (N-(3-dimethylaminopropyl)-N′-ethylcarbodiimide-HCl; 3.8 mg/ml) and filled up to 1 ml with MES buffer (pH 4.5).

Agitation is done overnight (around 10 h) at 4 degrees C. The particles are then cleaned by multiple centrifuging.

One prepares nanoparticles laden with cell lysate of Candida albicans wild type. Nanoparticles laden with cell lysate of Candida albicans TSA 1 knockout serve as the control.

### Preservation of the Protein Function in Nanoparticle Layers:

To stabilize the function of nanoparticle-bound trapping proteins in nanoparticle layers, the particles for the coating are suspended in 5% (w/v) aqueous trehalose solution.

### 1.3 Preparation of the Microarrays

To prepare fluorescently readable Candida diagnosis chips, the nanoparticles laden with Candida cell lysate are transferred by means of a Pin-Ring Spotter onto the pretreated glass substrate. The concentration of the particle suspensions used is 2% (w/v). Every needle contact with the surface transfers around 50 μl of suspension, and there are five pressings per spot. The spot diameter is around 150 μm. The placement of the individual spots on the substrate is freely programmable.

### 1.4 Use of the Candida Diagnosis Chips

### Preparation of Antibody:

Every 3 mg of the synthesized peptides HPGDETIKPS (SEQ ID NO: 2) and EASKEYFNKVNK (SEQ ID NO: 3) (20 mg of each synthesized), >70% purity; Thermo Electron Corporation, Ulm) in 3000 μl of PBS were coupled to 3 mg of Keyhole limpet hemocyanin (KLH, Sigma Aldrich, Taufkirchen) in 3000 μl water. The coupling was done at first at room temperature by fivefold addition each time of 2.4 μl of 5% glutaraldehyde (final concentration around 10 mmol/h) at intervals of 5 min. The reaction mix was incubated on ice for 30 minutes. The blocking was done with 24 μl of 1 mol/l glycine pH 8.5.

The coupled peptides were purified and half of each was used per animal. Two rabbits were immunized a total of four times at an interval of 30 days (Pineda, Berlin). Preimmune serum, serum of the immunization day 61, 90 and 120 was characterized.

Immobilized peptides for the affinity purification of the TSA1 antibodies were prepared by means of CNBr-activated sepharose 4B (Amersham Biosciences, Freiburg) according to the instructions of the company. 0.3 g of CNBr-activated sepharose 4B was placed in a test tube and allowed to swell for 15 min in 1 mmol/l of HCl, so that the beads were covered. After this, the sepharose was washed several times with a total of 300 ml of 1 mmol/l HCl and then with 7.5 ml of 100 mmol/l NaHCO3 0.5 mol/l NaCl pH 8.3 (binding buffer).

Every 2.5 mg of peptide 10 and peptide 12 were dissolved in 2 ml of binding buffer, added to the washed sepharose and incubated overnight at 4 degrees C. on the rotation wheel. Excess peptide was removed by onetime washing with 5 ml of binding buffer and the still remaining active groups were blocked with 1 mol/l of ethanol amine pH 8.0 for 2 h. The sepharose was alternatingly washed for at least three times with fivefold gel volume using 0.1 mol/l of Na-acetate 0.5 mol/l NaCl pH 4 and 0.1 mol/l of Tris-HCl 0.5 mol/l NaCl pH 8.0. The affinity matrix was washed another two times in PBS pH 7.4 and stored at 4 degrees C. with 0.02% (w/v) of azide.

### Making Contact With the Sample:

For the purifying, 3 ml of serum of the TSA 1 antibody was used. Incubation was done by rotation overnight at 4 degrees C., washing three times with PBS pH 7.4, then eluting with 0.1 mol/l of glycine pH 2.8. The eluate was collected in 1 ml fractions in 1.5 ml reaction vessels, in each of which 50 μl of 1 mol/l Tris-HCl pH 8.8 had been placed. In all, ten fractions were collected. These were measured in a quartz cell at 280 nm and the fractions 1-3 were purified and dialyzed against PBS pH 7.4. The dialysis was done once for 2 h and once overnight at 4 degrees C. in 2 liters of PBS each. The affinity purified and dialyzed TSA1 antibodies were combined with 0.02% (w/v) of azide and stored at 4 degrees C.

The nanoparticle surfaces are at first blocked for 1 h with a 3% (w/v) solution of BSA in PBS buffer. Then, incubation in the dark at room temperature is done for 1.5 h with a sample comprising purified anti-TSA 1 antibody (around 230 μmol/l or 5 μg per 100 ml of PBS+1% BSA). After that, washing is done in PBS for 30 min each.

The control is functional nanoparticles on which the cell lysate of a Candida strain is immobilized, which does not contain the antigen TSA 1, as the gene for this antigen has been disabled (knockout strain).

### Labeling of the Bound Anti-TSA 1 Antibodies:

The binding is detected with a fluorescently labeled secondary antibody against the species from which the antibodies are derived, in the animal experiment layout here: anti-rabbit antibodies (in the diagnostic test: anti-human antibodies). The fluorescently labeled secondary antibody is dissolved in a 1% BSA solution in PBS/Tween (0.1%) (0.7 μg per 100 ml). The chips are incubated with this for 1 h in the dark at room temperature and then washed for 30 min each in PBS/0.1% TritonX 100, in PBS and in MilliQ water. All steps are carried out in glass specimen slide stands.

### Reading of the Chips.

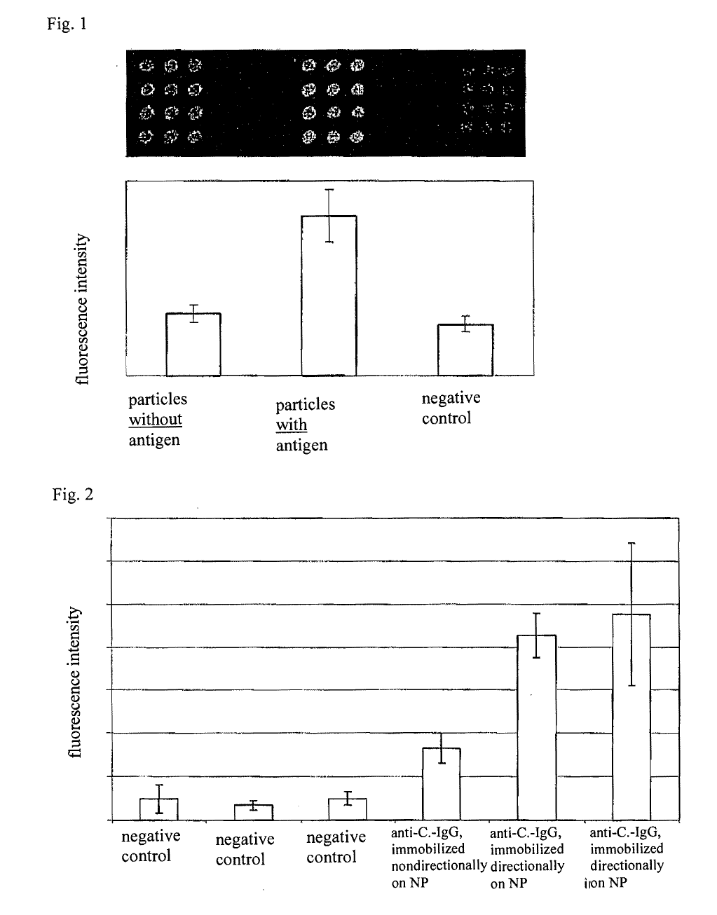

The fluorescence signal of the bound anti-Candida antibodies, anti-rabbit antibodies, is detected in a commercial chip reader system from the ArrayWorx company. The exposure times are between 0.1 s and 2 s and are kept constant within an experiment. The signal intensities are memorized in the form of gray scale levels. Evaluation of the data is done by means of the Aida program of the Raytest company, Berlin. The results are presented in FIG. 1.

### Example 2

### Detection of a Fluorescently Labeled Recombinant Candida albicans Antigen by Means of Candida Diagnosis Chips

The detection of the Candida specific antigen TSA 1 in a sample is carried out by immobilizing antibodies to TSA 1 on functional silica nanoparticles and depositing these bioactive nanoparticles as an affinity coating on a substrate. TSA 1 antigens present in the sample (in the experiment, for example, on chooses: TSA 1 maltose binding protein fusion construct (TSA 1-MPB)) bind to the anti-TSA 1 antibody, which is immobilized in three dimensional nanostructured affinity layers. In this example, recombinant fluorescently labeled TSA 1-MPB fusion protein was used as Candida antigen.

### 2.1 Preparation of Nanoparticle-Based Candida Diagnosis Chips

Corresponds to example 1.1.

### 2.2 Binding of the Molecule-Specific Recognition Sites to the Core/Shell Particles—Binding of Anti-TSA 1-IgG

The rabbit anti-TSA 1-IgG molecules used as an example can be bound a) nondirectionally and covalently to the functional nanoparticles or directionally via b) protein G or c) anti-rabbit IgG:

### a) Covalently, Nondirectionally:

1 mg of carboxy-functionalized silica particles is combined with 66 μl of rabbit anti-TSA 1 IgG solution (0.7 mg/ml) and 10 μl of an EDC solution (N-(3-dimethylaminopropyl)-N′-ethylcarbodiimide-HCl; 3.8 mg/ml) and filled up to 1 ml with MES buffer (pH 4.5). The mixture is agitated overnight at 4 degrees C., and then the particles are purified by multiple centrifugation.

### b) Via Proteing:

1 mg of carboxy-functionalized silica particles is combined with 10 μl of ProteinG Gamma Bind type 2 (Pierce) (3 mg/ml) and 10 μl of an EDC solution (N-(3-dimethylaminopropyl)-N′-ethylcarbodiimide-HCl; 3.8 mg/ml) and filled up to 1 ml with MES buffer (pH 4.5). The mixture is agitated overnight at 4 degrees C., and then the particles are purified by multiple centrifugation.

500 μg of ProteinG particles are combined with 26 μl of anti-TSA 1 IgG solution (0.7 mg/ml) and filled up to 500 μl with PBS. The mixture is agitated overnight at 4 degrees C., and then the particles are purified by multiple centrifugation.

### c) Via Anti-Rabbit IgG:

1 mg of carboxy-functionalized silica particles is combined with 66 μl of anti-rabbit IgG solution (0.7 mg/ml) and 10 μl of an EDC solution (N-(3-dimethylaminopropyl)-N′-ethylcarbodiimide-HCl; 3.8 mg/ml) and filled up to 1 ml with MES buffer (pH 4.5). The mixture is agitated overnight at 4 degrees C., and then the particles are purified by multiple centrifugation.

500 μg of anti-rabbit IgG particles are combined with 26 μl of anti-TSA 1 IgG solution (0.7 mg/ml) and filled up to 500 μl with PBS. The mixture is agitated overnight at 4 degrees C., and then the particles are purified by multiple centrifugation.

### Stabilization of the Protein Function:

To preserve/stabilize the protein function of the proteins bound to the nanoparticles in nanoparticle layers, the particles are suspended in 5% (w/v) aqueous trehalose solution for the coating.

### 2.3 Preparation of the Microarrays

Corresponds to example 1.3.

### 2.4 Use of the Candida Diagnosis Chips

### TSA 1—Maltose Binding Protein—Fusion Construct

For example, a fusion protein was used as the sample (TSA 1 antigen). The fusion protein (SEQ ID NO: 4) was cloned in order to perform the purification via maltose binding protein (MBP; SEQ ID NO: 5). TSA 1 (SEQ ID NO: 1) is connected to the C-terminal end of MBP (SEQ ID NO: 5) via a linker (SEQ ID NO: 6).

pMAL-p2X (NEB company) was used as the overexpression vector. The protein purification was carried out in familiar fashion according to the manufacturer's instructions.

Making Contact with the Sample:

The nanoparticle surfaces are at first blocked for 1 h with a 3% (w/v) solution of BSA in PBS buffer. They are then incubated at room temperature in the dark for 1 h with a solution of the fluorescently labeled recombinant TSA 1-MBP fusion protein antigen (40 μmol/l in PBS). The chips are then washed for 30 min each in PBS/0.1% TritonX 100, in PBS and in MilliQ water. All steps are carried out in glass specimen slide stands.

Anti-rabbit IgG, anti-goat IgG and/or streptavidine-coated nanoparticles are used as negative controls.

### Reading of the Chips:

See example 1.4. The results are presented in FIG. 2.

### Example 3

### Detection of Candida albicans Antigen by Means of Sandwich Technique on Nanoparticle-Based Candida Diagnosis Chip

The detection of Candida specific antigens in a sample is carried out by immobilizing antibodies to a TSA 1 on functional silica nanoparticles and depositing these bioactive nanoparticles as an affinity coating on a substrate. TSA 1 antigens present in the sample bind to the anti-Candida antibody, which is immobilized in the three dimensional nanostructured affinity layers. With the help of a fluorescently labeled detection antibody, the binding is detected (sandwich). Anti-goat IgG coated nanoparticles are used as negative controls.

### 3.1 Preparation of Nanoparticle-Based Candida Diagnosis Chips

Corresponds to example 1.1.

### 3.2 Binding of the Molecule-Specific Recognition Sites to the Core/Shell Particles—Binding of Rabbit Anti-Candida IgG

The binding of rabbit anti-Candida IgG to core/shell nanoparticles is done covalently, nondirectionally; corresponding to example 2.2. The proteins are stabilized as in example 2.2.

### 3.3 Preparation of the Microarrays

Corresponds to example 1.3.

### 3.4 Use of the Candida Diagnosis Chips

The anti-Candida nanoparticle surfaces are at first blocked for 1 h with a 3% (w/v) solution of BSA in PBS buffer and then incubated at room temperature for 1 h with a solution of the recombinant TSA 1-MBP fusion protein antigen (100 μmol/I in PBS). The chips are then washed for 30 min each in PBS/0.1% TritonX 100 and PBS, then blocked again for 30 min in BSA solution.

They are then incubated for 1 h in the dark at room temperature with a solution of the fluorescently labeled detection antibody (40 μmol/l in PBS) and finally washed for 30 min each in PBS/0.1% TritonX 100, in PBS and in MilliQ water. All steps are carried out in glass specimen slide stands.

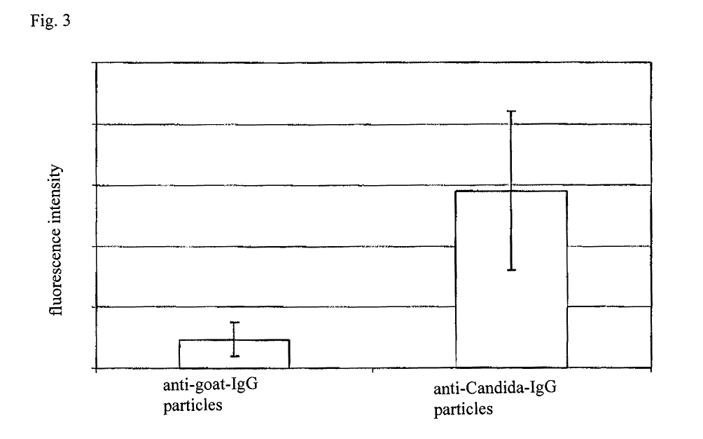

The results are presented in FIG. 3.

1. A Candida diagnosis chip, comprising a substrate with a surface and at least one microstructure arranged on the substrate surface with molecule-specific recognition sites immobilized thereon, wherein the molecule-specific recognition sites are selected from:
a) specific antibodies against protein TSA, and
b) protein TSA.

2. The Candida diagnosis chip of claim 1, wherein the microstructure is formed from at least two nanoparticles and the nanoparticles comprise the molecule-specific recognition sites.

3. The Candida diagnosis chip of claim 2, wherein the microstructure is formed from several three-dimensionally superimposed layers of nanoparticles with a thickness of 10 nm to 10 μm.

4. The Candida diagnosis chip of claim 1, wherein the microstructure is formed with inclusion of at least one protein stabilizing agent.

5. The Candida diagnosis chip of claim 1, wherein the substrate and/or the substrate surface is built up from metal, metal oxide, polymer, semiconductor material, glass, and/or ceramic.

6. The Candida diagnosis chip of claim 1, wherein the surface of the substrate is planar or prestructured, and the substrate is impermeable and/or porous.

7. The Candida diagnosis chip of claim 1, wherein one layer of a bonding agent is arranged between the substrate surface and the microstructure.

8. A method for the preparation of a Candida diagnosis chip, comprising the steps of:
a) preparing a substrate with a surface, and
b) depositing at least one microstructure on the surface of the substrate, wherein the microstructure contains at least two nanoparticles, on which are immobilized proteins with molecule-specific recognition sites selected from:
i) specific antibodies to the protein TSA, and
ii) protein TSA.

9. The method of claim 8, wherein step b) comprises the following substeps:
b1) functionalizing the surface of the nanoparticles with amino and/or carboxy functions, and
b2) immobilizing the proteins on the functionalized nanoparticles by bringing the proteins into contact with the functionalized nanoparticles.

10. A method for detecting Candida in clinical material, comprising the steps of:
a) preparing a sample of clinical material,
b) preparing a Candida diagnosis chip comprising a substrate with a surface and at least one microstructure arranged on the substrate surface with molecule-specific recognition sites immobilized thereon, wherein the molecule-specific recognition sites are selected from specific antibodies against protein TSA, and protein TSA; or obtaining a Candida diagnosis chip prepared by a method according to claim 8,
c) bringing the sample into contact with the Candida diagnosis chip under conditions which make possible a specific antigen/antibody binding, wherein Candida-specific molecules from the sample are bound specifically to the molecule-specific recognition sites of the Candida diagnosis chip, and
f) detecting the Candida-specific molecules bound specifically on the Candida diagnosis chip.

11. The method of claim 10, wherein nonbound Candida-specific molecules and nonspecific molecules are removed from the Candida diagnosis chip by washing with a biocompatible washing liquid in an additional step d).

12. The method of claim 10, wherein the detection method carried out in step f) is a fluorescence method.

13. The method of claim 12, wherein the Candida-specific molecules specifically bound on the Candida diagnosis chip are bound with fluorescently labeled molecules in an additional step e).

14. (canceled)

15. A kit for detecting Candida in clinical material, comprising: a Candida diagnosis chip comprising a substrate with a surface and at least one microstructure arranged on the substrate surface with molecule-specific recognition sites immobilized thereon, wherein the molecule-specific recognition sites are selected chosen from among specific antibodies against protein TSA, and protein TSA and/or a Candida diagnosis chip prepared by a method according to claim 8.

16. The Candida diagnosis chip of claim 3, wherein the thickness is 50 nm to 2.5 μm.

17. The Candida diagnosis chip of claim 16, wherein the thickness is 100 nm to 1.5 μm.


SEQUENCE LISTING (6 sequences)

SEQ ID NO: 1 - protein, 196 aa, Candida albicans
MAPVVQQPAPSFKKTAVVDGVFEEVTLEQYKGKWVLLAFIPLAFTFVCPSEIIAYSEAVKKFAEKDAQVLFASTDSEYTWLAWTNVARKDGGIGKVDFPVLADTNHSLSRDYGVLIEEEGVALRGIFLIDPKGVLRQITINDLPVGRSVEESLRLLEAFQFTEKYGEVCPANWHPGDETIKPSPEASKEYFNKVNK

SEQ ID NO: 2 - protein, 10 aa, Candida albicans
HPGDETIKPS

SEQ ID NO: 3 - protein, 12 aa, Candida albicans
EASKEYFNKVNK

SEQ ID NO: 4 - protein, 614 aa, Candida albicans
MKIKTGARILALSALTTMMFSASALAKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGIEGRISEFGSMAPVVQQPAPSFKKTAVVDGVFEEVTLEQYKGKWVLLAFIPLAFTFVCPSEIIAYSEAVKKFAEKDAQVLFASTDSEYTWLAWTNVARKDGGIGKVDFPVLADTNHSLSRDYGVLIEEEGVALRGIFLIDPKGVLRQITINDLPVGRSVEESLRLLEAFQFTEKYGEVCPANWHPGDETIKPSPEASKEYFNKVNK

SEQ ID NO: 5 - protein, 389 aa, Candida albicans
MKIKTGARILALSALTTMMFSASALAKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPAELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQT

SEQ ID NO: 6 - protein, 26 aa, Candida albicans
NSSSNNNNNNNNNNLGIEGRISEFGS